(19) United States
(12) Patent Application Publication
(10) Pub. No.: US 2024/0049987 A1
(43) Pub. Date: Feb. 15, 2024

(54) GAIT MEASUREMENT SYSTEM, GAIT MEASUREMENT METHOD, AND PROGRAM RECORDING MEDIUM

(71) Applicant: NEC Corporation, Minato-ku, Tokyo (JP)

(72) Inventors: Chenhui HUANG, Tokyo (JP); Kenichiro FUKUSHI, Tokyo (JP); Zhenwei WANG, Tokyo (JP)

(73) Assignee: NEC Corporation, Minato-ku, Tokyo (JP)

(21) Appl. No.: 17/766,308

(22) PCT Filed: Oct. 29, 2019
(86) PCT No.: PCT/JP2019/042363

§ 371 Date: Apr. 4, 2022

(51) Int. Cl. A61B 5/11; A61B 5/103; A61B 5/00

(52) U.S. Cl. CPC A61B 5/112; A61B 5/1038; A61B 5/7264; A61B 5/742

(57) ABSTRACT

A gait measurement system including: a data acquisition device that measures physical quantities related to pressures of left and right feet; and a calculation device that calculates a symmetry of walking using the physical quantities related to the pressures of the left and right feet.

### TECHNICAL FIELD

The present invention relates to a gait measurement system, a gait measurement method, and a program. In particular, the present invention relates to a gait measurement system for measuring a symmetry of walking, a gait measurement method, and a program.

### BACKGROUND ART

With increasing interest in healthcare for physical condition management, a technique for measuring a gait including features of walking of a pedestrian has been developed.

PTL 1 discloses a walking change determination device that includes an acceleration sensor and determines a change in walking of a user based on detected acceleration. The device of PTL 1 determines a degree of change, which is a degree of temporal change, based on the acceleration detected by the acceleration sensor and based on a temporal change in a trajectory during walking of a predetermined region to which the device is attached.

PTL 2 discloses a walking analysis system that calculates a stride length of a pedestrian using measurement data of sensors attached to a back of a foot, a lower leg, and a upper thigh of at least one of left and right feet of the pedestrian.

### CITATION LIST

### Patent Literature

[PTL 1] JP 5724237 B

[PTL 2] JP 5586050 B

### Non Patent Literature

[NPL 1] A. Segal, et al, “The Effect of Walking Speed on Peak Plantar Pressure,” Foot Ankle Int, 2004 25(12):926-33

[NPL 2] Y. Morio, et al, “The Relationship between Walking Speed and Step Length in Older Aged Patients,” Diseases 2019, 7, 17.

### SUMMARY OF INVENTION

### Technical Problem

When the device of PTL 1 is attached to a waist of a pedestrian, stride lengths of the left and right feet of the pedestrian can be calculated by specifying the positions of the feet from the projection of the measured waveform. However, in the method of PTL 1, the step size cannot be accurately calculated unless the lower limb is in a straight state. Therefore, in the method of PTL 1, the step size cannot be accurately calculated in case where an ankle joint is distorted.

According to the method of PTL 2, the sensor unit is attached to both feet, and waveforms of both feet can be measured by synchronizing measurement data of both feet. However, in the method of PTL 2, it is necessary to attach sensors to a plurality of positions on both feet, and thus, it is difficult to use the method on a daily basis.

It is important for healthcare to detect abnormality of walking of a pedestrian that affect measured data such as a stride length. From the viewpoint of abnormal detection of the walking, for example, there is a need to measure a symmetry of walking of a pedestrian as the gait of the pedestrian. When the symmetry of walking can be measured in real time, the abnormality occurring in a pedestrian can be found at an early stage. Therefore, a technique for measuring the symmetry of walking in day-to-day life is required. However, PTLs 1 and 2 do not disclose such a technique.

An object of the present invention is to solve the above-described problems and to provide a gait measurement system and the like capable of easily measuring a symmetry of walking in day-to-day life.

### Solution to Problem

According to an aspect of the present invention, a gait measurement system includes a data acquisition device configured to measure physical quantities related to pressures of both left and right feet, and a calculation device configured to calculate a symmetry of walking using the physical quantities related to the pressures of the left and right feet.

In a gait measurement method according to an aspect of the present invention, a computer acquires physical quantities related to pressures of left and right feet, and calculates a symmetry of walking using the acquired physical quantities related to the pressures of the left and right feet.

According to an aspect of the present invention, a program causes a computer to perform a process including acquiring physical quantities related to pressures of left and right feet and calculating a symmetry of walking using the acquired physical quantities related to the pressures of the left and right feet.

### Advantageous Effects of Invention

According to the present invention, it is possible to provide a gait measurement system and the like capable of easily measuring the symmetry of walking in day-to-day life.

### BRIEF DESCRIPTION OF DRAWINGS

FIG. 1 is a block diagram illustrating an example of a configuration of a gait measurement system according to a first example embodiment of the present invention.

FIG. 2 is a block diagram illustrating an example of a configuration of the data acquisition device of the gait measurement system according to the first example embodiment of the present invention.

FIG. 3 is a conceptual diagram illustrating an arrangement example of a pressure sensor of the data acquisition device of the gait measurement system according to the first example embodiment of the present invention.

FIG. 4 is a conceptual diagram illustrating an arrangement example of the data acquisition device of the gait measurement system according to the first example embodiment of the present invention.

FIG. 5 is a conceptual diagram for describing a coordinate system of sensor data acquired by the gait measurement system according to the first example embodiment of the present invention.

FIG. 6 is a conceptual diagram for describing an example of a walking parameter used by the gait measurement system according to the first example embodiment of the present invention.

FIG. 7 is a conceptual diagram for describing another example of the walking parameter used by the gait measurement system according to the first example embodiment of the present invention.

FIG. 8 is a conceptual diagram for describing a general gait cycle.

FIG. 9 is a graph for describing time-series data of pressure generated by the gait measurement system according to the first example embodiment of the present invention.

FIG. 10 is a block diagram for describing an example of a configuration of a calculation device of the gait measurement system according to the first example embodiment of the present invention.

FIG. 11 is a graph for describing an example of the time-series data of the pressure generated by the gait measurement system according to the first example embodiment of the present invention.

FIG. 12 is a flowchart for describing an example of an operation of a walking parameter calculation unit of the calculation device of the gait measurement system according to the first example embodiment of the present invention.

FIG. 13 is a flowchart for describing an example of an operation of a symmetry calculation unit of the calculation device of the gait measurement system according to the first example embodiment of the present invention.

FIG. 14 is a block diagram illustrating an example of a configuration of a gait measurement system according to a second example embodiment of the present invention.

FIG. 15 is a block diagram illustrating an example of a configuration of the calculation device of the gait measurement system according to the second example embodiment of the present invention.

FIG. 16 is a conceptual diagram for describing a position of marks attached to a periphery of a shoe when generating a regression model used by the gait measurement system according to the second example embodiment of the present invention.

FIG. 17 is a conceptual diagram for describing a walking line on which a pedestrian walks when generating the regression model used by the gait measurement system according to the second example embodiment of the present invention, and for describing an arrangement of a plurality of cameras for detecting the walking of the pedestrian.

FIG. 18 is a diagram illustrating a measurement result showing a relationship between a symmetry of pressures and a symmetry of step lengths generated with respect to walking of two subjects.

FIG. 19 is a flowchart for describing an example of an operation of a step length calculation unit of the gait measurement system according to the second example embodiment of the present invention.

FIG. 20 is a block diagram for describing an example of a configuration of a gait measurement system according to a third example embodiment of the present invention.

FIG. 21 is a conceptual diagram illustrating an example of information to be displayed on a display unit of a display device of the gait measurement system according to the third example embodiment of the present invention.

FIG. 22 is a flowchart for describing an example of an operation of the gait measurement system according to the third example embodiment of the present invention.

FIG. 23 is a conceptual diagram illustrating an example of a configuration of a gait measurement system according to a modified example of the third example embodiment of the present invention.

FIG. 24 is a conceptual diagram illustrating an example of information to be displayed on a display unit of a display device of the gait measurement system according to the modified example of the third example embodiment of the present invention.

FIG. 25 is a block diagram illustrating an example of a hardware configuration that achieves a calculation device according to each example embodiment of the present invention.

### EXAMPLE EMBODIMENTS

Hereinafter, example embodiments of the present invention will be described with reference to the drawings. However, example embodiments described below have technically preferable limitations for carrying out the present invention, but the scope of the invention is not limited to the following. In all the drawings used in the following description of the example embodiment, the same reference numerals are given to the same parts unless there is a particular reason. Further, in the following example embodiments, repeated description of similar configurations and operations may be omitted. In addition, directions of arrows in the drawings illustrate an example, and do not limit directions of signals between blocks.

### First Example Embodiment

First, a gait measurement system according to a first example embodiment of the present invention will be described with reference to the drawings. The gait measurement system according to the present example embodiment calculates a symmetry of walking using sensor data acquired by a sensor disposed on footwear such as a shoe. The symmetry of walking is an index representing a symmetry of a walking state of both feet during walking.

Hereinafter, an example will be described in which the gait measurement system calculates a walking parameter using sensor data acquired by a pressure sensor disposed on the footwear, and calculates the symmetry of walking using the calculated walking parameter. The walking parameter is a parameter calculated by using a physical quantity related to pressure such as foot pressure applied to the floor surface by the sole of foot.

### Configuration

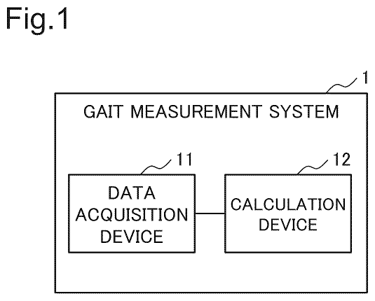

FIG. 1 is a block diagram illustrating a configuration of the gait measurement system 1 according to the present example embodiment. The gait measurement system 1 includes a data acquisition device 11 and a calculation device 12. The data acquisition device 11 and the calculation device 12 may be connected by a wired or wireless manner. In addition, the data acquisition device 11 and the calculation device 12 may be configured by a single device. The data acquisition device 11 may be excluded from the configuration of the gait measurement system 1, and only the calculation device 12 may constitute the gait measurement system 1.

The data acquisition device 11 is connected to the calculation device 12. The data acquisition device 11 includes a pressure sensor. For example, the data acquisition device 11 is installed on a user's footwear. The data acquisition device 11 converts a physical quantity related to pressure acquired by a pressure sensor into digital data (also referred to as sensor data), and transmits the converted sensor data to the calculation device 12.

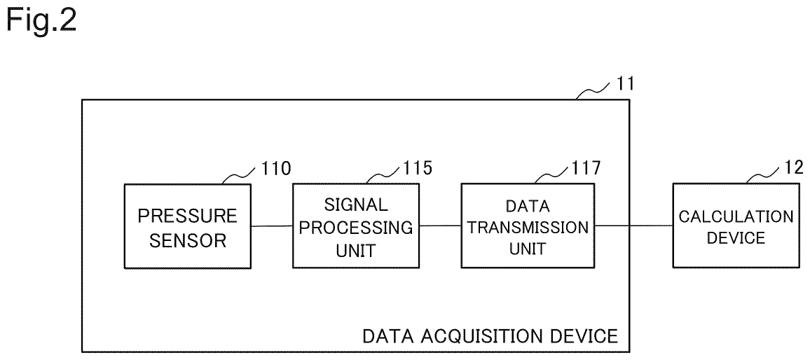

FIG. 2 is a block diagram illustrating an example of a detailed configuration of the data acquisition device 11. The data acquisition device 11 includes a pressure sensor 110, a signal processing unit 115, and a data transmission unit 117.

The pressure sensor 110 is a sensor that measures a physical quantity related to pressure. The pressure sensor 110 is connected to the signal processing unit 115. The pressure sensor 110 outputs a physical quantity related to the measured pressure to the signal processing unit 115.

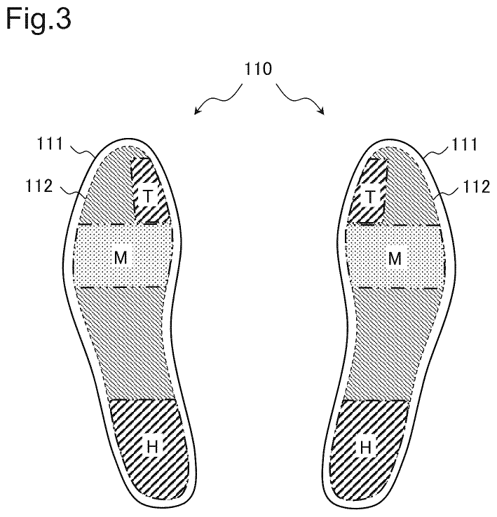

FIG. 3 is a conceptual diagram illustrating an example of the pressure sensor 110. The pressure sensor 110 includes a main body 111 and a sensor unit 112. The pressure sensor 110 is used in a state of being installed as an insole in a shoe. In FIG. 3, for use in the following description, a region in which an alphabet is added to a position T of a toe, a position H of a heel, and a position M of a medial plantar bulb (also referred to as a footrest) is illustrated.

The main body 111 has an outer shape of a footbed of a shoe. The main body 111 may have different shapes for the left foot and the right foot, or may have the same shape. Furthermore, the main body 111 may be made of a material of a general footbed, or may be made of a material having enhanced rigidity and functionality. For example, the main body 111 has a layered structure of at least two layers, and has a structure in which the sensor unit 112 is inserted between any layers or the sensor unit 112 is arranged on the surface.

The sensor unit 112 is installed inside or on a surface of the main body 111. The sensor unit 112 is connected to the signal processing unit 115 (not illustrated). The sensor unit 112 includes at least one sensor that measures a physical quantity related to pressure. The sensor unit 112 outputs the detected physical quantity to the signal processing unit 115.

For example, the sensor unit 112 detects a physical quantity related to pressure such as foot pressure and foot pressure distribution. For example, the sensor unit 112 can include a pressure sensor that detects pressure received from a sole of foot of a user wearing a shoe on which the data acquisition device 11 is installed. For example, the sensor unit 112 can include a sheet-like sensor sheet capable of measuring a pressure distribution. When a pressure sensor sheet is used as the sensor unit 112, the pressure distribution received from the sole can be measured. For example, the sensor unit 112 may be disposed at a specific position on the sole. For example, the sensor unit 112 may be disposed only at the position T of the toe and the position H of the heel. The sensor unit 112 may include a single sensor or may include a combination of a plurality of sensors. In a case where the sensor unit 112 includes a plurality of sensors, the sensor unit 112 may include a plurality of sensors of the same type, or may include a plurality of sensors of different types.

NPL 1 discloses an example showing that the relationship between the pressure by the sole of the foot and the walking speed varies depending on the position of the foot.

NPL 1: A. Segal, et al, “The Effect of Walking Speed on Peak Plantar Pressure,” Foot Ankle Int, 2004 25(12):926-33.

According to NPL 1, at least the pressure at the position T of the toe or the pressure at the position H of the heel show linearity with respect to the walking speed v. On the other hand, the pressure at the position M of the footrest portion does not show linearity with respect to the walking speed v when the walking speed increases.

The signal processing unit 115 is connected to the pressure sensor 110 and the data transmission unit 117. The signal processing unit 115 acquires a physical quantity related to pressure from the pressure sensor 110. The signal processing unit 115 converts the acquired physical quantity related to pressure into digital data, and outputs the converted digital data (also referred to as sensor data) to the data transmission unit 117. The sensor data includes at least pressure data converted into the digital data. The acquisition time of the data is associated with the pressure data. In addition, the signal processing unit 115 may be configured to output sensor data obtained by adding correction such as a mounting error, temperature correction, and linearity correction to the acquired pressure data.

The data transmission unit 117 is connected to the signal processing unit 115. In addition, the data transmission unit 117 is connected to the calculation device 12. The data transmission unit 117 acquires sensor data from the signal processing unit 115. The data transmission unit 117 transmits the acquired sensor data to the calculation device 12. The data transmission unit 117 may transmit the sensor data to the calculation device 12 via a wire such as a cable, or may transmit the sensor data to the calculation device 12 via wireless communication. For example, the data transmission unit 117 is configured to transmit the sensor data to the calculation device 12 via a wireless communicator capability (not illustrated) conforming to a standard such as Bluetooth (registered trademark) or Wi-Fi (registered trademark). The communicator capability of the data transmission unit 117 may conform to a standard other than Bluetooth (registered trademark) or Wi-Fi (registered trademark).

The data acquisition device 11 is achieved by, for example, an inertial measurement unit including the acceleration sensor and the angular velocity sensor, in addition to the pressure sensor. An example of the inertial measurement device is an inertial measurement unit (IMU). The IMU includes a triaxial acceleration sensor and a triaxial angular velocity sensor. Further, an example of the inertial measurement unit may include vertical gyro (VG). The VG has the same configuration as the IMU, and can output a roll angle and a pitch angle based on a direction of gravity by a technique called strap-down. Further, an example of the inertial measurement unit may include an attitude heading reference system (AHRS). The AHRS has a configuration in which an electronic compass is added to the VG. The AHRS can output a yaw angle in addition to the roll angle and the pitch angle. Further, an example of the inertial measurement unit may include a global positioning system/inertial navigation system (GPS/INS). The GPS/INS has a configuration in which the GPS is added to the AHRS. The GPS/INS may calculate a position in 3D space in addition to the roll angle, the pitch angle, and the yaw angle, and thus, can estimate a position with high accuracy. For example, the acceleration sensor and the angular velocity sensor are installed at positions relevant to the back side of the arch of the foot. Further, for example, the acceleration sensor or the angular velocity sensor may be fixed to a position of an ankle or a foot by a sock, a supporter, a band, or the like.

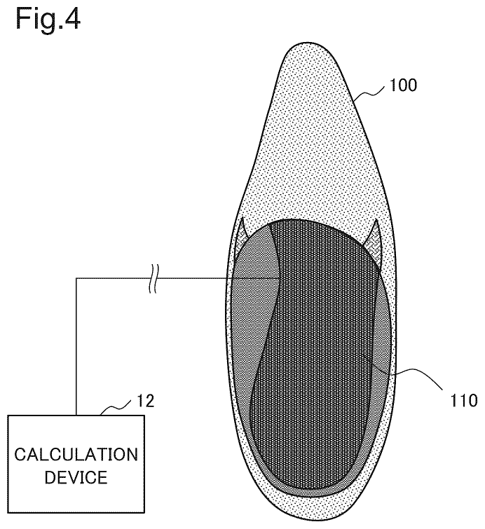

FIG. 4 is a conceptual diagram illustrating an example in which the pressure sensor 110 is installed in a shoe 100. For example, the pressure sensor 110 is disposed on the entire back surface of the foot. The pressure sensor 110 may be disposed on the entire back surface of the foot. For example, the pressure sensor 110 may be installed only at the position of the toe or the heel. In FIG. 4, the signal processing unit 115 and the data transmission unit 117 are omitted. The signal processing unit 115 and the data transmission unit 117 are achieved by a microcomputer (not illustrated) having a communication function.

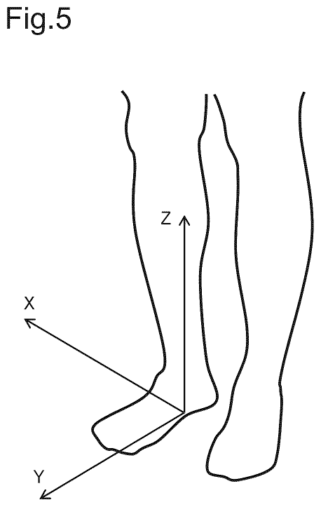

FIG. 5 is a conceptual diagram for describing a coordinate system (X axis, Y axis, Z axis) set for a foot of a pedestrian. FIG. 3 illustrates an example in which a lateral direction of a pedestrian is set to an X-axis direction (rightward direction is positive), a traveling direction of the pedestrian is set to a Y-axis direction (forward direction is positive), and a direction of gravity is set to a Z-axis direction (vertically upward direction is positive).

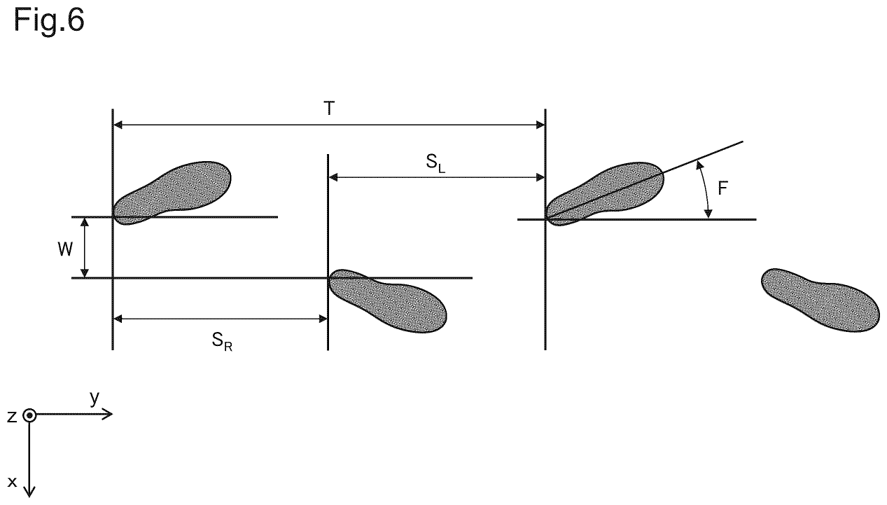

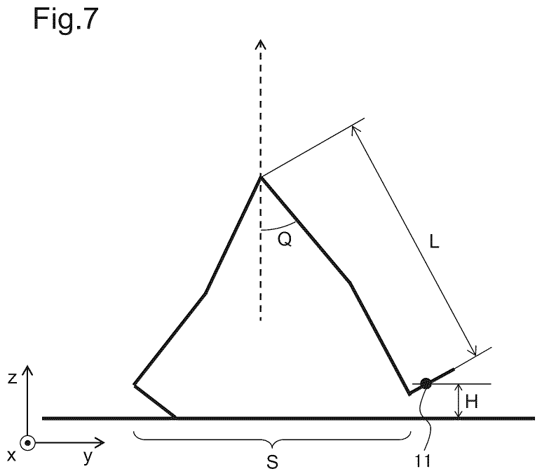

Here, the walking parameters other than pressure will be described with some examples. FIGS. 6 and 7 are conceptual diagrams for describing an example of the walking parameters.

FIG. 6 illustrates a step length SR of a right foot, a step length SL of a left foot, a stride length T, a step width W, and a foot angle F. The step length SR of the right foot is a distance of one step of the right foot. In FIG. 6, the step length SR of the right foot is a difference in a Y coordinate between the heel of the right foot and the heel of the left foot when a state in which a sole of the left foot is grounded transitions to a state in which the heel of the right foot swung out in the traveling direction is landed. The step length SL of the left foot is a distance relevant to one step of the left foot. In FIG. 6, the step length SL of the left foot is a difference in the Y coordinate between the heel of the right foot and the heel of the left foot when a state in which a sole of the right foot is grounded transitions to a state in which the heel of the right foot swung out in the traveling direction is landed. The stride length T is a distance of two steps. The stride length T is a sum of the step length SR of the right foot and the step length SL of the left foot. The step width W is an interval between the right foot and the left foot. In FIG. 6, the step width W is a difference between an X coordinate of a center line of the heel of the right foot in the grounded state and an X coordinate of a center line of the heel of the left foot in the grounded state, in one step. The foot angle F is an angle formed by the center line of the foot and the traveling direction (Y axis) in the state where the back surface of the foot is grounded.

FIG. 7 illustrates a forefoot angle Q, a lower limb length L, and a sensor height H. The forefoot angle Q is also expressed as forward foot placement relative to the trunk (FFP), and is an angle formed by a central axis of a thigh of a leg swung out forward and the direction of gravity (Z axis). The lower limb length L is a length of a leg of a pedestrian. The sensor height H is a height of the data acquisition device 11 with respect to a floor plane (XY plane). Hereinafter, the floor plane is also referred to as a horizontal plane.

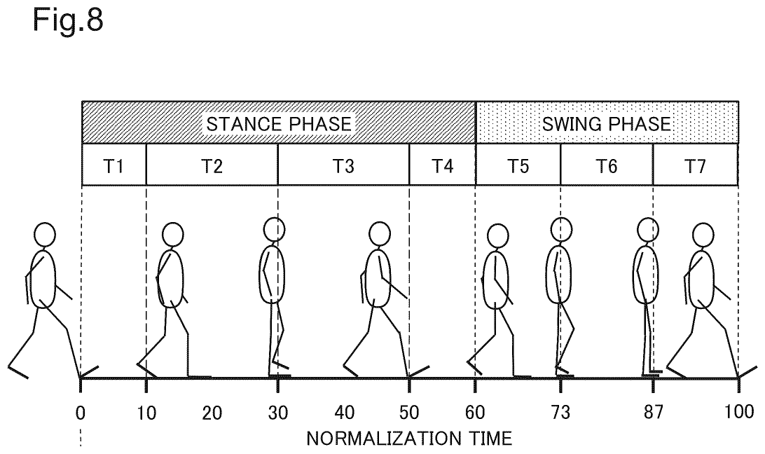

Here, an acquisition timing of pressure data used by the gait measurement system 1 will be described with reference to the drawings. FIG. 8 is a conceptual diagram for describing a gait cycle of a general pedestrian. A horizontal axis in FIG. 8 represents time (also referred to as normalization time) normalized with one gait cycle of one foot as 100%. A stance phase is further subdivided into a loading response period T1, a mid-stance period T2, a terminal stance period T3, and a pre-swing period T4. In addition, a swing phase is further classified into an initial swing period T5, a mid-swing period T6, and a terminal swing period T7.

Generally, one gait cycle of one foot is largely divided into the stance phase in which at least a part of the back side of the foot is in contact with the ground and the swing phase in which the back side of the foot is separated from the ground. Immediately after the heel of the pedestrian touches the ground, the pressure received by the sensor unit 112 from the heel becomes maximum. A peak at which the pressure received from the heel becomes maximum is referred to as a first peak. On the other hand, immediately before the toe of the pedestrian is separated from the ground, the pressure received by the sensor unit 112 from the toe becomes maximum. A peak at which the pressure received from the toe becomes maximum is referred to as a second peak. When the positive and negative pressures are opposite depending on how the data acquisition device 11 is attached, the maximum and minimum of the pressure are exchanged.

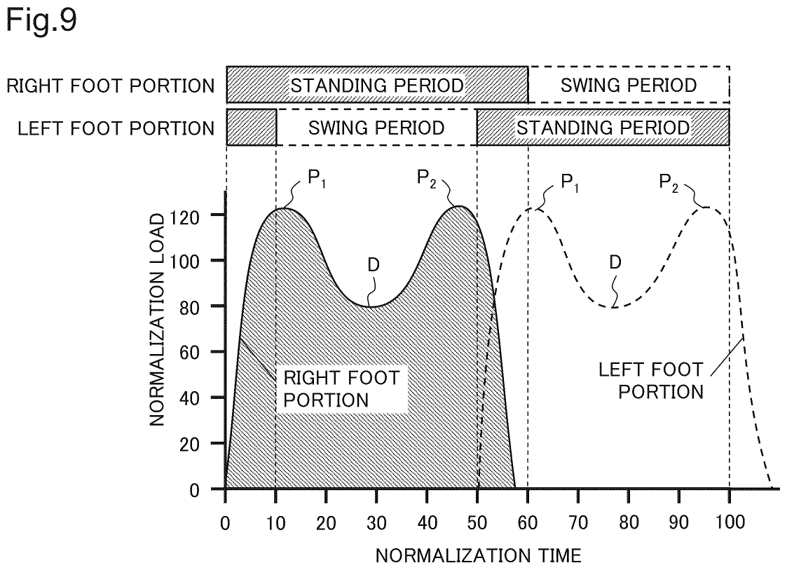

FIG. 9 is a graph illustrating an example of a temporal change in foot pressure (pressure received from the sole of the foot) measured when a human walks. The horizontal axis in FIG. 9 is a normalization time obtained by normalizing the lapse of time associated with walking of a human, and is relevant to the horizontal axis in FIG. 8. In a curve illustrated in FIG. 9, a solid line indicates a temporal transition of foot pressure in a right foot portion, and a broken line indicates a temporal transition of foot pressure in a left foot portion.

In a temporal transition (solid line) of a vertical component force of the right foot portion during walking, two mountains (a first peak P1, second peak P2) and one valley (dip D) appear. For example, the first peak P1, the second peak P2, and the dip D can be separated into waveforms indicated by each of the peaks. The first peak P1 is caused by an impact when the entire sole of the foot comes into contact with a ground by an ankle joint vertical rotational motion after the heel of the right foot is grounded. The second peak P2 is caused by the pressure applied to the ground by the toe of the right foot during the forward attitude of the heel grounding of the left foot and the toe taking off of the right foot that occurs between the terminal stance period and the pre-swing period of the right foot. A value of a foot pressure at an apex of the second peak P2 is relevant to a value obtained by adding a load by a weight and a vertical component of a force generated by a muscle when a pedestrian moves forward. The dip D is caused by the acceleration in the direction opposite to the gravity caused by the upward motion of the left foot generated in the middle of the standing foot of the right foot.

The calculation device 12 is connected to the data acquisition device 11. In addition, the calculation device 12 is connected to an external system or device (not illustrated). The calculation device 12 receives sensor data from the data acquisition device 11. The calculation device 12 calculates the symmetry of walking using the received sensor data. The calculation device 12 outputs information on the calculated symmetry of walking to the external system or device (not illustrated).

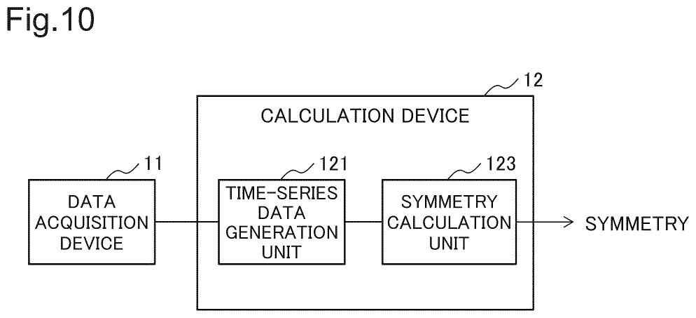

FIG. 10 is a block diagram illustrating an example of a configuration of the calculation device 12. The calculation device 12 includes a time-series data generation unit 121 and a symmetry calculation unit 123.

The time-series data generation unit 121 is connected to the data acquisition device 11. In addition, the time-series data generation unit 121 is connected to the symmetry calculation unit 123. The time-series data generation unit 121 acquires the pressure data from the data acquisition device 11 with respect to the left and right feet. The time-series data generation unit 121 synchronizes data according to the acquisition time of the pressure data in the data acquisition device 11 installed in the left and right shoes, and generates the time-series data of the pressure values of both feet using the pressure data. The time-series data generation unit 121 outputs the generated time-series data of the pressure values of both feet to the symmetry calculation unit 123.

The symmetry calculation unit 123 is connected to the time-series data generation unit 121. In addition, the symmetry calculation unit 123 is connected to an external system or device (not illustrated). The symmetry calculation unit 123 acquires the time-series data of the pressure values of the left and right feet from the time-series data generation unit 121. The symmetry calculation unit 123 calculates the symmetry of walking using the time-series data of the pressure values of the left and right feet. For example, the symmetry calculation unit 223 calculates the symmetry of the pressures applied by each of the left and right feet as the symmetry of walking. The symmetry calculation unit 223 may calculate an arithmetic mean or a geometric mean of the symmetry of the pressures as the symmetry of walking. The symmetry calculation unit 123 outputs information on the calculated symmetry of walking to the external system or device (not illustrated).

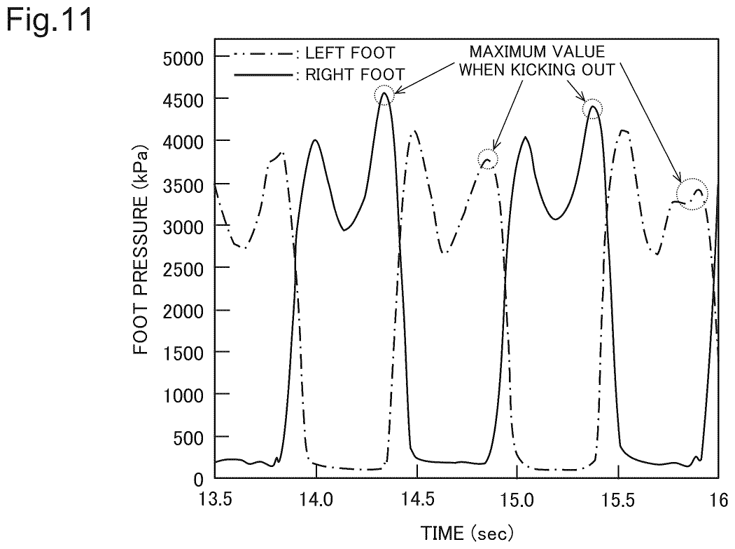

FIG. 11 is a graph illustrating an example of time-series data of pressure applied to a sole of foot of a pedestrian who simulatively walks with asymmetrical left-right walking. FIG. 11 illustrates an example in which the step length SL of the left foot is larger than that of the step length SR of the right foot. In FIG. 11, the time-series data of the pressure of the right foot is shown by a solid line, and the time-series data of the attitude angle of the left foot is shown by a dashed-dotted line. Referring to FIG. 11, when the right foot (solid line) and the left foot (dashed-dotted line) are compared, the difference of the second peak of two maximum peaks (first peak, second peak) of each time-series data is large. In the example of FIG. 11, since the left foot greatly kicks forward as compared with the right foot, the pressure by the right foot, which is an axial foot at the time of kicking out the left foot, is larger. That is, the value of the second peak is larger in the right foot than in the left foot. On the other hand, for the first peak, the difference between the left and right feet is small. Therefore, the second peak is more suitable as an index for evaluating the symmetry of walking than the first peak.

For example, the symmetry calculation unit 123 acquires the time-series data of the pressure values of both feet from the time-series data generation unit 121. The symmetry calculation unit 123 detects the maximum peak from the time-series data of the pressure values of both feet. From the time-series data of the pressure for one step, a first maximum peak (first peak) and a second maximum peak (second peak) following the first peak are detected.

For example, the symmetry calculation unit 123 calculates the symmetry SIp of the pressures by using the pressure value of the second peak at which the difference between the left and right sides increases in a case where walking is asymmetric. For example, the calculation device 12 calculates the symmetry SIp of the pressures using the following Equation 1.

SIp=(P_{2R}−P_{2L})/(P_{2R}+P_{2L}) (1)

In the above Equation 1, each of P_{2R} and P_{2L} is pressure values of the second peaks of the right foot and the left foot.

For example, the symmetry calculation unit 123 may calculate the symmetry SIp of the pressures using the pressure values of both the first peak and the second peak. For example, the calculation device 12 calculates the symmetry SIp of the pressures using the following Equations 2 or 3.

SIp=P_{2R}/P_{1R}−P_{2L}/P_{1L} (2)

SIp=P_{2R/}P_{1R}+P_{2L}/P_{1L} (3)

In Equations 2 and 3 above, each of P_{1R} and P_{1L} is pressure values at the first peak of each of the right foot and the left foot.

The configuration of the gait measurement system 1 of the present example embodiment has been described above. The configurations of FIGS. 1 to 4 and 10 are examples, and the configuration of the gait measurement system 1 of the present example embodiment is not limited to the configurations of FIGS. 1 to 4 and 10.

For example, the gait measurement system 1 can be achieved by the pressure sensor 110, a microcomputer including a part of the functions (signal processing unit 115, data transmission unit 117) of the data acquisition device 11, and the calculation device 12. In addition, for example, the gait measurement system 1 can be achieved by the pressure sensor 110, a microcomputer including a part of the functions (signal processing unit 115, data transmission unit 117) of the data acquisition device 11, and a mobile terminal or a server including the calculation device 12. The time-series data generation unit 121 and the symmetry calculation unit 123 constituting the calculation device 12 may be distributed to different devices. For example, the time-series data generation unit 121 may be included in the microcomputer, and the symmetry calculation unit 123 may be included in the mobile terminal or server.

### Operation

Next, an example of an operation of the calculation device 12 of the present example embodiment will be described with reference to the drawings. Hereinafter, the operations of the time-series data generation unit 121 and the symmetry calculation unit 123 included in the calculation device 12 will be individually described.

### Time-Series Data Generation Unit

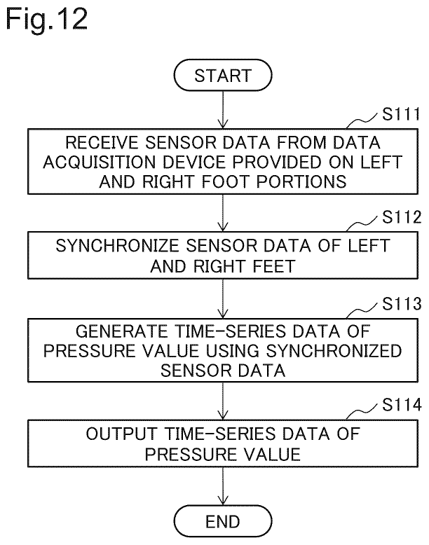

FIG. 12 is a flowchart for describing an example of the operation of the time-series data generation unit 121 of the calculation device 12. In the following description along the flowchart of FIG. 12, the time-series data generation unit 121 is an operation subject.

In FIG. 12, first, the time-series data generation unit 121 receives the sensor data (pressure data) of the left and right feet from each of the data acquisition devices 11 installed in the left and right foot portions (step S111).

Next, the time-series data generation unit 121 synchronizes the sensor data of the left and right feet (step S112).

Next, the time-series data generation unit 121 generates the time-series data of the pressure values of the left and right feet using the synchronized sensor data of the left and right feet (step S113).

Then, the time-series data generation unit 121 outputs the generated time-series data of the pressure values of the left and right feet to the symmetry calculation unit 123 (step S114).

### Symmetry Calculation Unit

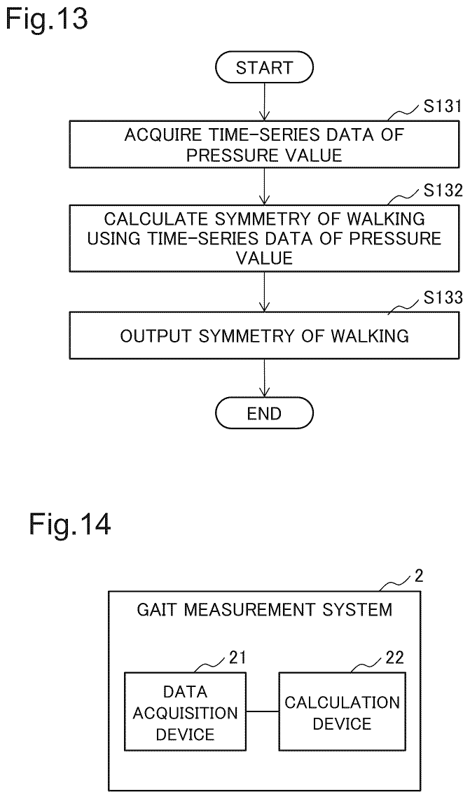

FIG. 13 is a flowchart for describing an example of an operation of the symmetry calculation unit 123 of the calculation device 12. In the following description along the flowchart of FIG. 13, the symmetry calculation unit 123 is an operation subject.

In FIG. 13, first, the symmetry calculation unit 123 acquires the time-series data of the pressure values of the left and right feet from the time-series data generation unit 121 (step S131).

Next, the symmetry calculation unit 123 calculates the symmetry of the pressures as the symmetry of walking using the acquired time-series data of the pressure values of the left and right feet (step S132).

Then, the symmetry calculation unit 123 outputs the calculated symmetry of walking (step S133).

An example of the operation of the calculation device 12 of the present example embodiment has been described above. The flowcharts of FIGS. 12 and 13 are examples, and the operation of the calculation device 12 of the present example embodiment is not limited to the processing along the flowcharts of FIGS. 12 and 13.

As described above, the gait measurement system according to the present example embodiment includes a data acquisition device configured to measure physical quantities related to pressures of both left and right feet, and a calculation device configured to calculate a symmetry of walking using the physical quantities related to the pressures of the left and right feet.

The gait measurement system according to one aspect of the present example embodiment includes a time-series data generation unit and a symmetry calculation unit. The time-series data generation unit generates the time-series data of the pressure values using the physical quantities related to the pressures of the left and right feet. The symmetry calculation unit calculates the symmetry of the pressures of the left and right feet as the symmetry of walking using the time-series data of the pressure values of the left and right feet. According to the present example embodiment, it is possible to easily measure the symmetry of walking in day-to-day life.

In addition, in one aspect of the present example embodiment, the symmetry calculation unit calculates the symmetry of walking using the relationship of maximum values of peaks relevant to each other between the left and right feet in one gait cycle among at least one peak appearing in each of the time-series data of the pressure values of the left and right feet. In addition, in one aspect of the present example embodiment, the symmetry calculation unit calculates the symmetry of walking using the relationship of maximum values of peaks relevant to each other between the left and right feet in one gait cycle among at least one peak appearing in each of the time-series data of the pressure values of the left and right feet.

For example, the symmetry calculation unit calculates the symmetry of walking using at least one of the maximum value of the first peak, the maximum value of the second peak, and the maximum value of the dip appearing between the first peak and the second peak, among at least one peak appearing in each of the time-series data of the pressure values of the left and right feet. The first peak is a peak at which a pressure received from a heel of a pedestrian becomes maximum. The second peak is a peak at which a pressure received from a toe of a pedestrian becomes maximum. For example, the symmetry calculation unit calculates the symmetry of walking using the relationship between the maximum value of the first peak, the maximum value of the second peak, and the maximum value of the dip.

According to one aspect of the present example embodiment, it is possible to accurately measure the symmetry of walking using the physical quantity related to the pressure measured by the data acquisition device installed on the footwear such as the shoe without using a large-scale device. That is, according to one aspect of the present example embodiment, it is possible to accurately measure the symmetry of walking in day-to-day life.

### Second Example Embodiment

Next, a gait measurement system according to a second example embodiment of the present invention will be described with reference to the drawings. The gait measurement system of the present example embodiment is different from the gait measurement system of the first example embodiment in that step lengths are calculated from a symmetry of walking parameters by applying a regression model that associates the symmetry of the walking parameters with a symmetry of step lengths. Hereinafter, the description of the same configuration or operation as those of the first example embodiment may be omitted.

### Configuration

FIG. 14 is a block diagram schematically illustrating a configuration of a gait measurement system 2 according to the present example embodiment. The gait measurement system 2 includes a data acquisition device 21 and a calculation device 22. The data acquisition device 21 and the calculation device 22 may be connected by a wired or wireless manner. In addition, the data acquisition device 21 and the calculation device 22 may be configured by a single device. The data acquisition device 21 may be excluded from the configuration of the gait measurement system 2, and only the calculation device 22 may constitute the gait measurement system 2.

The data acquisition device 21 is connected to the calculation device 22. The data acquisition device 21 includes a pressure sensor. The data acquisition device 21 converts a physical quantity related to pressure acquired by a pressure sensor into digital data (also referred to as sensor data), and transmits the converted sensor data to the calculation device 22. The data acquisition device 21 has a configuration relevant to the data acquisition device 11 of the first example embodiment.

The calculation device 22 is connected to the data acquisition device 21. In addition, the calculation device 22 is connected to an external system or device (not illustrated). The calculation device 22 receives sensor data from the data acquisition device 21. The calculation device 22 calculates the symmetry of walking using the received sensor data. The calculation device 22 calculates the symmetry of the step lengths of both feet from the calculated symmetry of the walking using the regression model that associates the symmetry of the walking with the symmetry of the step lengths. Further, the calculation device 22 calculates the step lengths of both feet using the calculated symmetry of the step lengths of both feet. The calculation device 22 outputs the calculated step lengths of both feet to an external system or device (not illustrated).

For example, the calculation device 22 uses a general-purpose regression model generated using data of a plurality of subjects. For example, the calculation device 22 uses a regression model generated using data of a plurality of subjects having a similar walking tendency (disease or injury, nature, etc.). For example, the calculation device 22 uses a regression model that is generated individually.

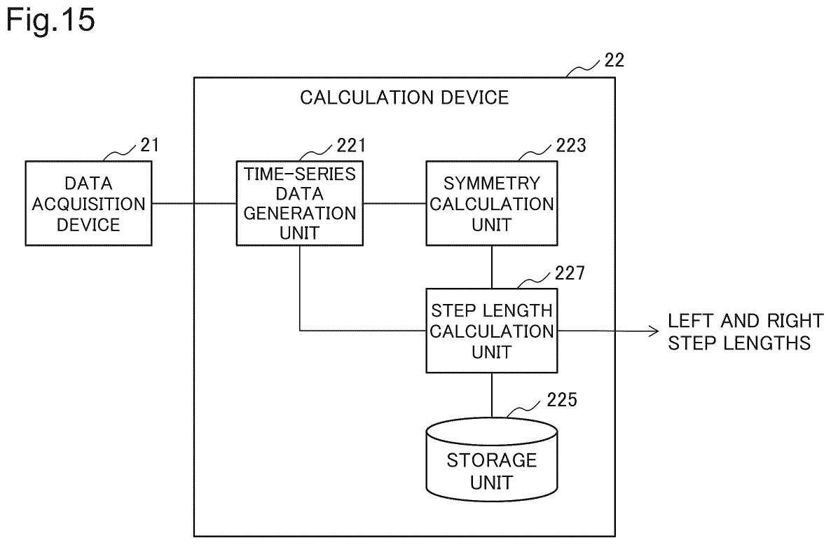

FIG. 15 is a block diagram illustrating an example of a configuration of the calculation device 22. The calculation device 22 includes a time-series data generation unit 221, a symmetry calculation unit 223, a storage unit 225, and a step length calculation unit 227.

The time-series data generation unit 221 is connected to the data acquisition device 21. In addition, the time-series data generation unit 221 is connected to the symmetry calculation unit 223 and the step length calculation unit 227. The time-series data generation unit 221 acquires the sensor data including the pressure data from the data acquisition device 21 with respect to the left and right feet. The time-series data generation unit 121 synchronizes the acquired pressure data with both the left and right feet to generate the time-series data of the pressure values of both feet. The time-series data generation unit 221 outputs the generated time-series data of the pressure values of both feet to the symmetry calculation unit 223 and the step length calculation unit 227. The time-series data generation unit 221 has a configuration relevant to the time-series data generation unit 121 of the first example embodiment.

The symmetry calculation unit 223 is connected to the time-series data generation unit 221 and the step length calculation unit 227. The symmetry calculation unit 223 acquires the time-series data of the pressure values of both feet from the time-series data generation unit 221. The symmetry calculation unit 223 calculates the symmetry of the pressures as the symmetry of walking using the time-series data of the pressure values of both feet. The symmetry calculation unit 223 may calculate an arithmetic mean or a geometric mean of the symmetry of the pressures as the symmetry of walking. The symmetry calculation unit 223 outputs the calculated symmetry of the pressures to the step length calculation unit 227. The symmetry calculation unit 223 has a configuration relevant to the symmetry calculation unit 123 of the first example embodiment.

The storage unit 225 is connected to the step length calculation unit 227. The storage unit 225 stores the regression model that associates the symmetry of the pressures with the symmetry of the step lengths. The regression model may be a universal model registered in advance in the gait measurement system 2, or may be individual models for each pedestrian.

The step length calculation unit 227 is connected to the time-series data generation unit 221, the symmetry calculation unit 223, and the storage unit 225. In addition, the step length calculation unit 227 is connected to an external system or device (not illustrated). The step length calculation unit 227 acquires the symmetry of the pressures from the symmetry calculation unit 223. The step length calculation unit 227 calculates the symmetry of the step lengths by applying the acquired symmetry of the pressures to the regression model stored in the storage unit 225. Further, the step length calculation unit 227 acquires the time-series data of the pressure values from the time-series data generation unit 221. The step length calculation unit 227 calculates the stride length of the pedestrian using the acquired time-series data of the pressure values. The step length calculation unit 227 calculates each of the step length of the right foot and the step length of the left foot using the calculated symmetry of the step lengths and the step lengths. The step length calculation unit 227 outputs each of the calculated step lengths of left and right feet.

The configuration of the gait measurement system 2 of the present example embodiment has been described above. The configurations of FIGS. 14 and 15 are one example, and the configuration of the gait measurement system 2 of the present example embodiment is not limited to the configurations of FIGS. 14 and 15.

For example, the gait measurement system 2 can be achieved by the pressure sensor 210 and the IMU including a part of the data acquisition device 21 and the calculation device 22. In addition, for example, the gait measurement system 2 can be achieved by the pressure sensor 210, the IMU including a part of the data acquisition device 21, and a mobile terminal or a server including the calculation device 22.

For example, the time-series data generation unit 221, the symmetry calculation unit 223, the storage unit 225, and the step length calculation unit 227 constituting the calculation device 22 may be distributed to different devices. For example, the time-series data generation unit 221 may be included in the IMU, and the symmetry calculation unit 223, the storage unit 225, and the step length calculation unit 227 may be included in the mobile terminal or the server. In addition, for example, the time-series data generation unit 221 may be included in the IMU, and at least any one of the symmetry calculation unit 223, the storage unit 225, and the step length calculation unit 227 may be included in different mobile terminal or server. In addition, the storage unit 225 may be stored in a storage accessible from the step length calculation unit 227 included in the mobile terminal or the server.

### Regression Model

Next, an example of generating the regression model using the relationship between the symmetry of the pressure and the symmetry of the step lengths will be described.

As illustrated in FIG. 9, in the time-series data for one gait cycle of the foot pressure (normalized load) of the left and right feet, a first peak P1, a dip D, and a second peak P2 appear in order. In addition, as illustrated in FIG. 11, when the walking becomes asymmetric, the mutual relationship of pressures at the first peak P1, the dip D, and the second peak P2 collapses. Focusing on this correlation, it is estimated that there is some relationship between the pressure value (Hereinafter, also referred to as a peak pressure value) of the peak such as the first peak P1 or the dip D, and the second peak P2 and the step length of the pedestrian. The peak pressure value is one of the walking parameters.

Here, the hypothesis that the step length S can be linearly regressed by the relationship of the following Equation 5 using the regression model f(F) having the walking parameter F, such as the first peak P1, the dip D, and the second peak P2 appearing in each of the left and right feet, as a variable is established.

S=C×f(F) (5)

In Equation 5, C denotes a coefficient.

The regression model f(F) is a model generated using the relationship between the walking parameters such as the first peak P1 or the dip D and the second peak P2 and the symmetry of the step length. The coefficient C has individual differences depending on a weight or a walking speed. In the present example embodiment, the calculation equation of Equation 5 is compared with a calculation equation for calculating a step length S by another approach, and a parameter not depending on individual differences included in a calculation equation of another approach is set as the regression model f(F).

NPL 1 discloses an example in which the pressure (FIG. 3) at the position T of the toe and the position H of the heel show linearity with respect to the walking speed. According to NPL 1, the pressure at the position T of the toe or the pressure at the position H of the heel shows linearity with respect to the walking speed v. On the other hand, the pressure at the position M of the footrest portion does not show linearity with respect to the walking speed. That is, based on NPL 1, by using the pressure at the position T of the toe or the position H of the heel, linearity can be obtained between the walking speed and the pressure up to a relatively high walking speed.

Here, it is assumed that linearity is satisfied between the walking speed of the pedestrian and the pressures at the position T of the toe and the position H of the heel based on NPL 1. Based on this assumption, as in the following Equations 6 and 7, a peak value PT of the pressure at the position T of the toe and a peak value PH of the pressure at the position H of the heel position H are associated with the weight w of the pedestrian and the walking speed v.

PT=k₁×w×v+b₁ (6)

PH=k₂×w×v+b₂ (7)

In the above Equations 6 and 7, k₁ and k₂ are relevant to inclinations, and b₁ and b₂ are relevant to intercept.

The walking speed v of the pedestrian can be calculated by using the following Equation 8 or Equation 9 obtained by modifying the above Equations 6 and 7.

v=(PT−b₁)/k₁/W (8)

v=(PH−b₂)/k₂/W (9)

The weight w, the inclination k₁, and the inclination k₂ are stored in advance in the storage unit 225 or a database (not illustrated).

Then, the stride length T can be calculated from the walking speed v by using the following Equation 10.

T=v×t (10)

In Equation 10, t is a time of one gait cycle. For example, the time interval of the continuous first peak P1, the time interval of the second peak P2, and the time interval of the dip D of one foot are relevant to t.

Since the foot pressure is related to the weight w, it cannot be calculated using the same calculation expression for pedestrians with different weights w. In addition, since the foot pressure is related to the walking speed v, the foot pressure cannot be calculated using the same calculation expression when the walking state is different even for the same person. Therefore, in the present example embodiment, as described later, in order to exclude individual differences or differences in the walking state, the step length is calculated using the symmetry of the step calculated using the symmetry of the pressure without using the components of the weight w and the walking speed v.

NPL 2 discloses an example in which the ratio of the step lengths to the height of the foot and the walking speed has a proportional relationship.

NPL 2: Y. Morio, et al, “The Relationship between Walking Speed and Step Length in Older Aged Patients,” Diseases, 2019 March; 7(1):17.

FIG. 2 of NPL 2 discloses an example showing that a ratio of a step length to a height of foot and a maximum value of a walking speed have a proportional relationship regardless of individual differences.

Assuming that a height of a foot of a pedestrian depends on a lower limb length L of a pedestrian, it is estimated that there is a relationship (proportional relationship) represented by the following Equation 11 between a ratio S/L of the step length S to the lower limb length L and the walking speed v based on NPL 2.

S/L=k×v (11)

In Equation 11, L denotes the lower limb length and k denotes the proportionality constant.

Here, the relationship of the following Equation 12 is derived based on Equations 5 and 11.

C×f(F)=k×v×L (12)

In the right side of Equation 12, the walking speed v and the lower limb length L depend on individual differences, and the proportional constant k does not depend on individual differences. That is, the coefficient C is relevant to a product of the walking speed v and the lower limb length L depending on individual differences, and the regression model f(F) is relevant to a proportional coefficient k not depending on individual differences.

In general, a symmetry SIs of the step length S is calculated by the following Equation 13.

SIs=(S_{R}−S_{L})/(S_{R}+S_{L}) (13)

In the above Equation 13, each of S_{R} and S_{L} is the step lengths of the right foot and the left foot.

The step length (S_{R} and S_{L}) of the right and left foot in the above equation 13 includes the walking speed v and the lower limb length L depending on individual differences. Therefore, in the present example embodiment, the symmetry SIs of the step length S is calculated using the regression model that does not depend on individual differences. Specifically, as described below, the symmetry SIs of the step lengths S is calculated using the symmetry SIp of the pressures calculated using the regression model f(P1, P2, D) (see Equations 14 to 18 below).

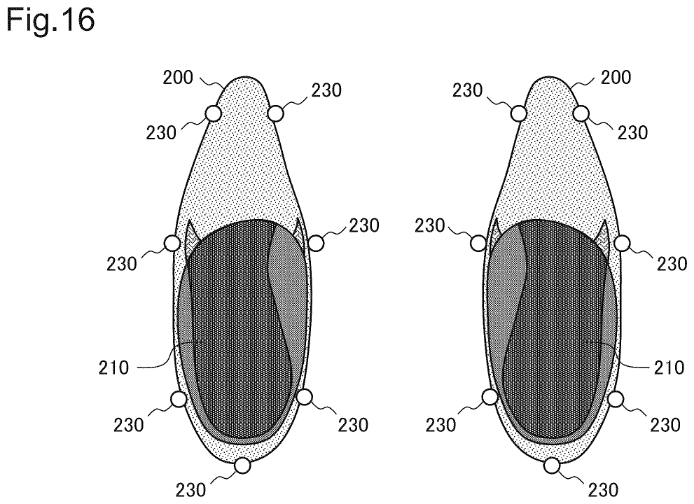

Here, an example of a specific method of generating a regression model will be described with reference to FIGS. 16 to 18. In the examples of FIGS. 16 to 18, marks for motion capture are attached to shoes, and a trajectory of foot of a pedestrian who walks while wearing the shoes is captured by a camera and the foot pressure of the pedestrian is measured, thereby generating the regression model.

FIG. 16 illustrates an example in which a plurality of marks 230 for motion capture are attached to the shoes 200 of both feet. In the example of FIG. 16, a total of 7 marks 230, 3 marks on each of the left and right side surfaces and 1 mark on the heel side surface, are attached to each of the shoes 200 of both feet. Attachment positions of the plurality of marks 230 illustrated in FIG. 16 are examples, and the attachment positions of the plurality of marks 230 are not limited to the positions illustrated in FIG. 16. Further, FIG. 16 illustrates an example in which the pressure sensor 210 is installed inside the shoe 200, but the pressure sensor 210 does not have to be installed in the shoe 200 for motion capture.

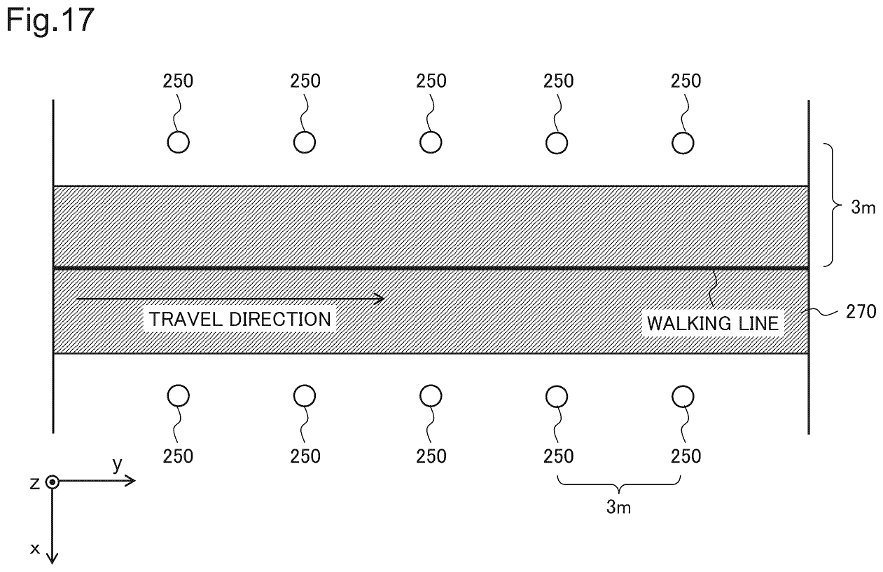

FIG. 17 is a conceptual diagram illustrating an example of a walking line when motion capturing walking of a pedestrian wearing the shoes 200 to which the plurality of marks 230 are attached, and arrangement positions of a plurality of cameras 250. A sheet-shaped pressure sensor 270 is arranged on a walking surface on which a pedestrian walks. The plurality of cameras 250 and pressure sensors 270 are connected to a computer (not illustrated) that generate the regression model. In the example of FIG. 17, five cameras 250 (10 cameras in total) are arranged on each side across the walking line. Each of the plurality of cameras 250 is arranged at a height of 2 m from a horizontal plane (XY plane) and at a position of 3 m from the walking line at intervals of 3 m, focusing on the walking line on which the pedestrian walks.

In the example of FIG. 17, for a pedestrian walking in a traveling direction (Y direction) along a walking line, a moving image captured by a plurality of cameras 250 and a physical quantity related to the pressure detected by the pressure sensor 270 are obtained. By associating the step lengths of the left and right feet of the pedestrian obtained from the image with the symmetry calculated using the pressure applied to the pressure sensor 270, it is possible to generate the regression model that associates the symmetry of the pressures with the symmetry of the step lengths.

The movements of the plurality of marks 230 installed on the shoes 200 of the pedestrian walking along the walking line can be analyzed using moving images captured by the plurality of cameras 250. By considering the plurality of marks 230 as one rigid body and analyzing the movement of their center of gravity, it is possible to generate a regression model that relates the symmetry of the pressures and the symmetry of the step lengths.

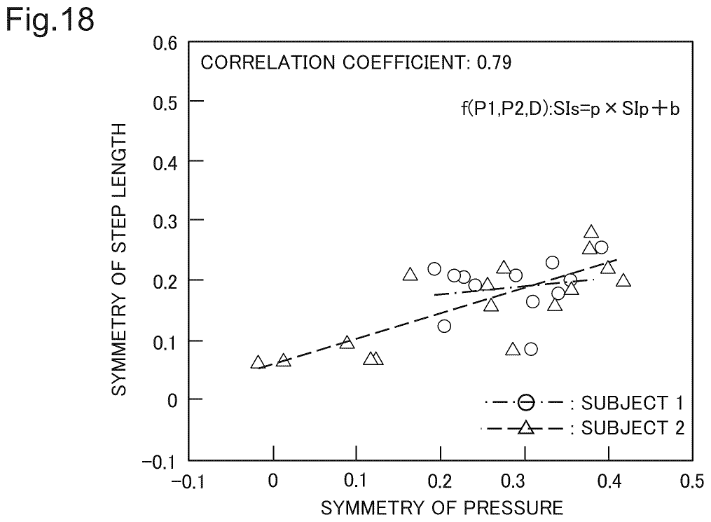

FIG. 18 illustrates an example of the relationship between the symmetry SIp of the pressure and the symmetry SIs of the step length obtained by motion capturing walking of two subjects (subject 1, subject 2). In the example of FIG. 18, the symmetry SIp of the pressures is calculated using the following Equation 14.

SIp=(P1−D)/(P2−D) (14)

Equation 14 is an empirical formula in which the high correlation is obtained by verifying the relationship between the symmetry of various pressures obtained by combining the peak pressure values P1, P2, and D, that are the walking parameters and the symmetry of the step lengths. The symmetry SIp of the pressures may be calculated for one of the left and right feet.

For the subject 1, Linearity (dashed-dotted line) was obtained by linear regression of the plot (∘) in which the symmetry SIp of the pressures is associated with the symmetry SIs of the step lengths. In addition, also for the subject 2, linearity (broken line) was observed when a plot (Δ) of the symmetry SIp of the pressures and the symmetry SIs of the step length was linearly regressed. That is, the regression model indicating the relationship between the symmetry SIp of the pressures and the symmetry SIs of the step length can be individually generated for each pedestrian. When such a regression model is used, the regression models for each pedestrian may be stored in advance in the storage unit 225.

In addition, for the two subjects (subject 1, subject 2), the correlation coefficient of the straight line obtained by linearly regressing the plots (∘ and Δ) of the symmetry SIp of the pressures and the symmetry SIs of the step lengths were linearly regressed was 0.79. This shows the possibility that the regression model indicating the relationship between the symmetry SIp of the pressures and the symmetry SIs of the step lengths can be used as a universal model regardless of the subject. When such a regression model is used, the existing regression model may be stored in advance in the storage unit 225 regardless of the pedestrian. For example, the regression model f(P1, P2, D) of the following Equation 15 in which a relational expression between the symmetry SIp of the pressures and the symmetry SIs of the step lengths obtained from the walking of the plurality of subjects is collected may be stored in the storage unit 225 in advance.

f(P1, P2, D):SIs=p×SIp +b (15)

In the above Equation 15, p denotes a proportional constant, and b denotes an intercept.

Since the sum of the step length S_{R} of the right foot and the step length S_{L} of the left foot is relevant to a stride length T (Equation 16), the difference between the step length S_{R} of the right foot and the step length S_{L} of the left foot can be expressed as the following Equation 17.

S_{R}+S_{L}=T (16)

S_{R}−S_{L}=T×SIs (17)

That is, each of the step length S_{R} of the right foot and the step length S_{L} of the left foot is put together in the following relational expression 18.

$$\{ \begin{matrix}{S}_{R} = \frac{T ( SIs + 1 )}{2} \\ {S}_{L} = \frac{T ( 1 - SIs )}{2}\end{matrix} \qquad ( 18 )$$

Hereinafter, the above Expression 18 is referred to as a relational expression U.

The step length calculation unit 227 calculates the step length T using Expressions 8 to 10. In addition, the step length calculation unit 227 calculates the symmetry SIs of the step lengths S by applying the symmetry SIp of the pressures calculated from the sensor data measured by the data acquisition device 21 to the regression model. The step length calculation unit 227 calculates each of the step length S_{R} of the right foot and the step length S_{L} of the left foot by substituting the symmetry SIs of the step length S and the stride length T into the relational expression U (Expression 18). The step length calculation unit 227 may calculate the stride length T by performing second-order integration on the acceleration measured by the sensor (not illustrated) installed in the shoe of one of the left and right feet.

An example of generating the regression model using the relationship between the symmetry of the pressures and the symmetry of the step lengths will be described above. The method of generating the regression model is an example, and the method of generating the regression model used by the gait measurement system 2 of the present example embodiment is not limited.

### Operation

Next, an example of an operation of the calculation device 22 of the present example embodiment will be described with reference to the drawings. Hereinafter, since the operations of each of the time-series data generation unit 221 and the symmetry calculation unit 223 included in the calculation device 22 is similar to which of the first example embodiment, only the operation of the step length calculation unit 227 will be described.

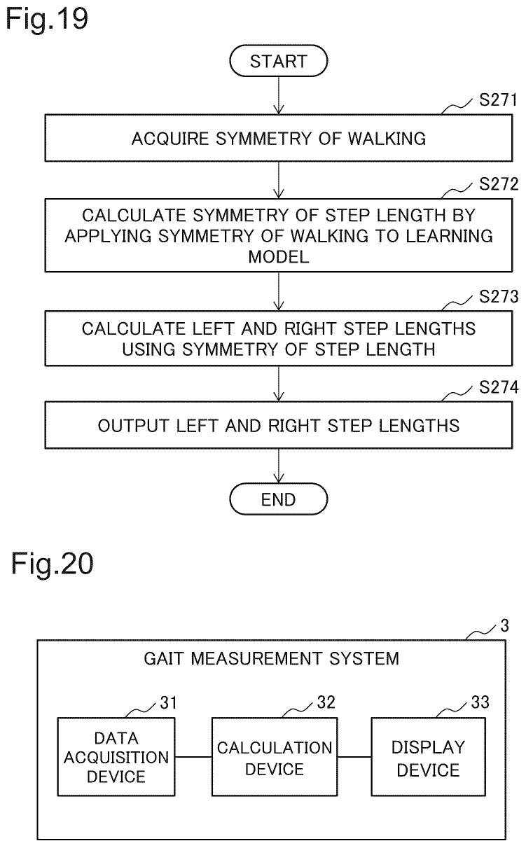

FIG. 19 is a flowchart for describing an example of the operation of the step length calculation unit 227. In the following description along the flowchart of FIG. 19, the step length calculation unit 227 is the main operation.

In FIG. 19, first, the step length calculation unit 227 acquires the symmetry of the walking (symmetry of pressures) from the symmetry calculation unit 223 (step S271).

Next, the step length calculation unit 227 calculates the symmetry of the step lengths by applying the symmetry of walking to the regression model (step S272).

Next, the step length calculation unit 227 calculates the step lengths of the left and right feet using the calculated symmetry of the step lengths (step S273).

Then, the step length calculation unit 227 outputs the calculated step lengths of the left and right feet (step S274).

An example of the operation of the step length calculation unit 227 of the calculation device 22 of the present example embodiment has been described above. The flowchart of FIG. 19 is an example, and the operation of the step length calculation unit 227 of the present example embodiment is not limited to the processing along the flowchart of FIG. 19.

As described above, the gait measurement system of the present example embodiment includes the calculation device including the storage unit and the step length calculation unit in addition to the time-series data generation unit and the symmetry calculation unit. The storage unit stores a regression model in which the symmetry of walking calculated using the relationship between the maximum value of the first peak, the maximum value of the second peak, and the maximum value of the dip is associated with the symmetry of the step length. The step length calculation unit calculates the symmetry of the step lengths from the symmetry of walking using the regression model, and calculates the step lengths of the left and right feet using the calculated symmetry of the step length.

According to the present example embodiment, it is possible to accurately measure the step lengths of the left and right feet by using the physical quantity related to the pressure measured by the data acquisition device installed on the footwear such as the shoe without using a large-scale device. That is, according to one aspect of the present example embodiment, it is possible to accurately measure the step lengths of the left and right feet in day-to-day life. In addition, in the present example embodiment, by using the regression model having the generality of the symmetry of walking, it is also possible to reduce the time and effort to generate the regression model again at the time of using the system.

### Third Example Embodiment

Next, a gait measurement system according to a third example embodiment of the present invention will be described with reference to the drawings. A gait measurement system of the present example embodiment is different from the gait measurement systems of the first and second example embodiments in that the gait measurement system includes a display device that displays information on a symmetry of walking. Hereinafter, the configuration in which the display device is added to the gait measurement system of the second example embodiment will be exemplified, and description of the same configuration and operation as those of the second example embodiment may be omitted.

### Configuration

FIG. 20 is a block diagram schematically illustrating a configuration of a gait measurement system 3 according to the present example embodiment. The gait measurement system 3 includes a data acquisition device 31, a calculation device 32, and a display device 33. The data acquisition device 31, the calculation device 32, and the display device 33 may be connected by a wired or wireless manner. In addition, the data acquisition device 31, the calculation device 32, and the display device 33 may be configured by a single device.

The data acquisition device 31 is connected to the calculation device 32. The data acquisition device 31 includes a pressure sensor. The data acquisition device 31 converts a physical quantity related to pressure acquired by a pressure sensor into digital data (also referred to as sensor data), and transmits the converted sensor data to the calculation device 32. The data acquisition device 31 has a configuration relevant to the data acquisition device 21 of the second example embodiment.

The calculation device 32 is connected to the data acquisition device 31 and the display device 33. The calculation device 32 receives sensor data from the data acquisition device 31. The calculation device 32 calculates the symmetry of walking using the received sensor data. The calculation device 32 calculates the symmetry of the step lengths of both feet from the calculated symmetry of the walking using the regression model that associates the symmetry of the walking with the symmetry of the step lengths. Further, the calculation device 32 calculates the step lengths of both feet using the calculated symmetry of the step lengths of both feet. The calculation device 32 outputs the calculated step length of both feet to the display device 33.

The display device 33 is connected to the calculation device 32. The display device 33 acquires information on the step lengths of the left and right feet or the symmetry of the step lengths from the calculation device 32. The display device 33 causes the display unit of the display device 33 to display information on the acquired step lengths of the left and right feet or the symmetry of the step lengths.

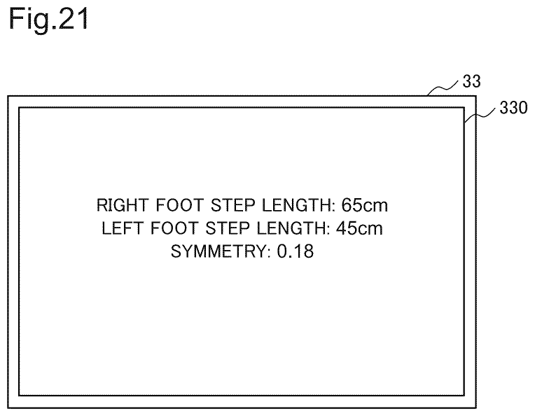

FIG. 21 illustrates an example in which the information on the step lengths of the left and right feet or the symmetry of the step lengths is displayed on the display unit 330 of the display device 33. The example of FIG. 21 is an example in which information indicating that the step length of the right foot is 65 cm, the step length of the right foot is 45 cm, and the symmetry of the left and right feet is 0.18 is displayed on the display unit 330 of the display device 33.

As illustrated in FIG. 21, a user who visually recognizes the information displayed on the display unit 330 of the display device 33 can estimate a walking state of a pedestrian according to the information displayed on the display unit 330. The information displayed on the display unit 330 is not limited to the example of FIG. 21 as long as the information is relevant to the step lengths of the left and right feet and the symmetry of the step lengths.

The outline of the configuration of the gait measurement system 3 of the present example embodiment has been described above. The configuration of FIG. 20 is an example, and the gait measurement system 3 of the present example embodiment is not limited to the configuration of FIG. 20.

For example, the gait measurement system 3 can be achieved by the pressure sensor, the IMU including a part of the data acquisition device 31 and the calculation device 32, and the mobile terminal or computer including the display device 33. In addition, for example, the gait measurement system 3 can be achieved by the pressure sensor, the IMU including a part of the data acquisition device 31, and the mobile terminal or computer including the calculation device 32 and the display device 33. In addition, for example, the gait measurement system 3 can be achieved by the IMU including a part of the data acquisition device 31, a server including the calculation device 32, and the mobile terminal or the computer including the display device 33.

### Operation

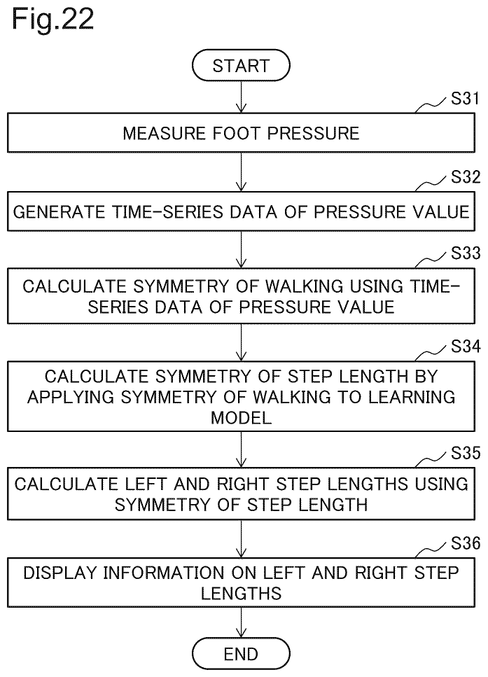

Next, an operation of the gait measurement system 3 according to the present example embodiment will be described with reference to the drawings. FIG. 22 is a flowchart for describing an example of the operation of the gait measurement system 3. In the following description along the flowchart of FIG. 22, the gait measurement system 3 is an operation subject.

In FIG. 22, first, the gait measurement system 3 measures the foot pressure (step S31).

Next, the gait measurement system 3 generates the time-series data of the pressure value using the pressure data for several steps (step S32).

Next, the gait measurement system 3 calculates the symmetry of walking (symmetry of pressure) using the time-series data of the pressure value (step S33).

Next, the gait measurement system 3 calculates the symmetry of the step lengths by applying the calculated symmetry of walking to the regression model (step S34).

Next, the gait measurement system 3 calculates the step lengths of the left and right feet using the calculated symmetry of the step lengths (step S35).

Then, the gait measurement system 3 displays the information on the step lengths of the left and right feet or the symmetry of the step lengths on the display unit 330 of the display device 33 (step S36).

An example of the operation of the gait measurement system 3 of the present example embodiment has been described above. The flowchart of FIG. 22 is an example, and the operation of the gait measurement system 3 of the present example embodiment is not limited to the processing along the flowchart of FIG. 22.

### Modified Example

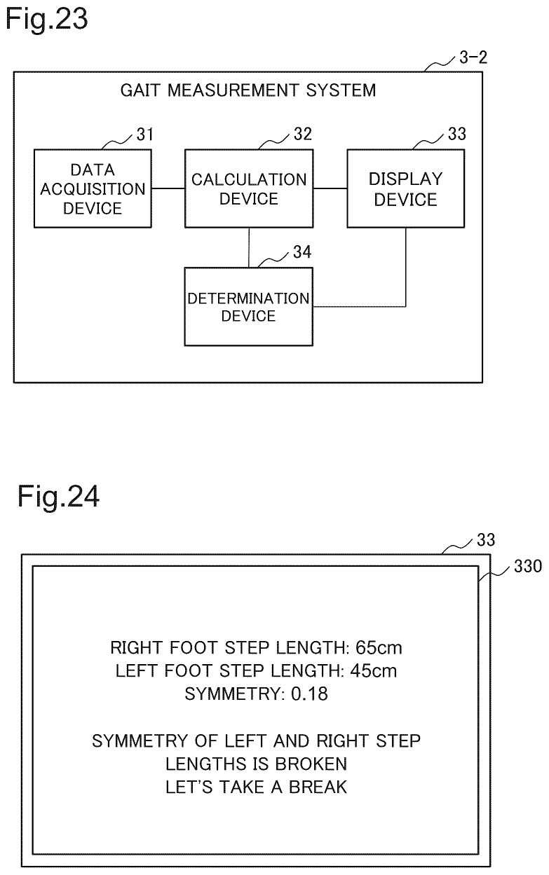

Next, a modified example of the present example embodiment will be described with reference to the drawings. FIG. 23 is a block diagram illustrating an example of a configuration of a gait measurement system 3-2 according to the modified example. The gait measurement system 3-2 of FIG. 23 is different from the gait measurement system 3 of FIG. 20 in that the gait measurement system 3-2 includes a determination device 34. Configurations of each of a data acquisition device 31, a calculation device 32, and a display device 33 of the gait measurement system 3-2 in FIG. 23 are similar to the configurations of the gait measurement system 3 in FIG. 20, and thus a detailed description thereof will be omitted.

The determination device 34 is connected to the calculation device 32 and the display device 33. The determination device 34 acquires information on step lengths of left and right feet or a symmetry of step lengths from the calculation device 32. The determination device 34 determines values of the step lengths of the left and right feet or values of the symmetry of the step lengths according to a magnitude relationship with a preset threshold. The determination device 34 outputs, to the display device 33, determination results related to the values of the step lengths of the left and right feet or the values of the symmetry of the step lengths. The determination results regarding the values of the step lengths of the left and right feet and the values of the symmetry of the step lengths are displayed on the display unit 330 of the display device 33.

For example, the determination device 34 makes a determination regarding energy cost, pain, muscle weakness, a degree of recovery from stroke due to rehabilitation, and the like of a pedestrian according to the magnitude relationship with the preset threshold value or a difference from the threshold value. For example, a plurality of threshold values may be set, and the determination results may be prepared for each area determined by the plurality of threshold values. The determination device 34 generates the display information according to the relationship between the determination result and the threshold value, and outputs the display information to display device 33.

FIG. 24 illustrates an example in which the values of the step lengths of the left and right feet, the value of the symmetry of the step lengths, and the determination results are displayed on the display unit 330 of the display device 33 as the information on the step lengths of the left and right feet and the symmetry of the step lengths. The example of FIG. 24 is an example in which information indicating that the step length of the right foot is 65 cm, the step length of the left foot is 45 cm, and the symmetry of the left and right feet is 0.18 is displayed on the display unit 330 of the display device 33. Furthermore, in the example of FIG. 24, based on the value of the symmetry, a determination result of “the symmetry of the left and right step lengths has collapsed” or an advice of “Let's take a little break” according to the determination result is displayed on the display unit 330.

As illustrated in FIG. 24, a user who visually recognizes the information displayed on the display unit 330 of the display device 33 can estimate a walking state of a pedestrian according to the information displayed on the display unit 330. The information displayed on the display unit 330 is not limited to the example of FIG. 24 as long as the information is relevant to the step lengths of the left and right feet and the symmetry of the step lengths.

As described above, the gait measurement system of the present example embodiment includes the display device that displays the information on the symmetry of walking. According to the present example embodiment, the walking state of the pedestrian can be estimated by referring to the information on the symmetry of walking displayed on the display device.

### Hardware

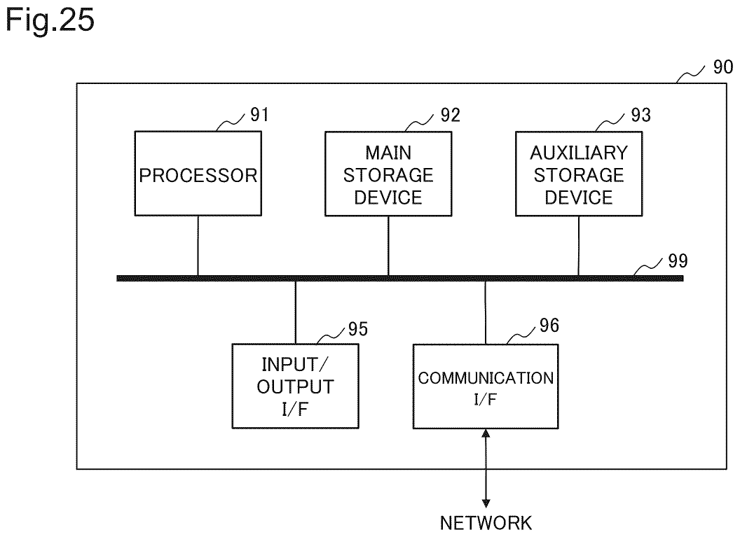

Here, the hardware configuration for achieving the calculation device according to each example embodiment of the present invention will be described by taking the information processing device 90 (also referred to as a computer) of FIG. 25 as an example. The information processing device 90 in FIG. 25 is a configuration example for executing the processing of the calculation device and the like of each example embodiment, and does not limit the scope of the present invention.

As illustrated in FIG. 25, the information processing device 90 includes a processor 91, a main storage device 92, an auxiliary storage device 93, an input/output interface 95, and a communication interface 96. In FIG. 25, the interface is abbreviated as I/F (interface). The processor 91, the main storage device 92, the auxiliary storage device 93, the input/output interface 95, and the communication interface 96 are data-communicably connected to each other via a bus 99. In addition, the processor 91, the main storage device 92, the auxiliary storage device 93, and the input/output interface 95 are connected to a network such as the Internet or an intranet via the communication interface 96.

The processor 91 expands the program stored in the auxiliary storage device 93 or the like to the main storage device 92, and executes the expanded program. In the present example embodiment, a software program installed in the information processing device 90 may be used. The processor 91 executes the processing by the calculation device according to the present example embodiment.

The main storage device 92 has an area in which the program is developed. The main storage device 92 is achieved by, for example, a volatile memory such as a dynamic random access memory (DRAM). Furthermore, a nonvolatile memory such as a magnetoresistive random access memory (MRAM) may be configured/added as the main storage device 92.

The auxiliary storage device 93 stores various data. The auxiliary storage device 93 is configured by a local disk such as a hard disk or a flash memory. Various data may be stored in the main storage device 92, and the auxiliary storage device 93 may be omitted.

The input/output interface 95 is an interface for connecting the information processing device 90 and peripheral devices. The communication interface 96 is an interface for connecting to an external system or device through a network such as the Internet or an intranet based on a standard or a specification. The input/output interface 95 and the communication interface 96 may be shared as an interface connected to an external device.

The information processing device 90 may be configured to connect input devices such as a keyboard, a mouse, or a touch panel, if necessary. These input devices are used to input information and settings. When a touch panel is used as the input device, a display screen of the display device may also serve as the interface of the input device. Data communication between the processor 91 and the input device may be mediated by the input/output interface 95.

In addition, the information processing device 90 may be provided with the display device for displaying information. When the display device is provided, the information processing device 90 preferably includes a display control device (not illustrated) for controlling the display of the display device. The display device may be connected to the information processing device 90 via the input/output interface 95.

The information processing device 90 may be equipped with a disk drive, if necessary. The disk drive is connected to the bus 99. The disk drive mediates reading a data/program from the recording medium, writing the processing result of the information processing device 90 to the recording medium, and the like between the processor 91 and a recording medium (program recording medium) (not illustrated). The recording medium can be achieved by, for example, an optical recording medium such as a compact disc (CD) or a digital versatile disc (DVD). Further, the recording medium may be achieved by a semiconductor recording medium such as a universal serial bus (USB) memory or a secure digital (SD) card, a magnetic recording medium such as a flexible disk, or other recording media.

The above is an example of a hardware configuration for achieving the calculation device according to each example embodiment of the present invention. The hardware configuration of FIG. 25 is an example of a hardware configuration for achieving the calculation device according to each example embodiment, and does not limit the scope of the present invention. In addition, the program for causing a computer to execute the processing related to the calculation device according to each example embodiment is also included in the scope of the present invention. Furthermore, the program recording medium in which the program according to each example embodiment is recorded is also included in the scope of the present invention.

The components of the calculation device of each example embodiment may be arbitrarily combined. In addition, the components such as the calculation device of each example embodiment may be achieved by software or may be achieved by a circuit.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

### REFERENCE SIGNS LIST

1, 2, 3 gait measurement system

11, 21, 31 data acquisition device

12, 22, 32 calculation device

33 display device

34 determination device

115 signal processing unit

117 data transmission unit

121, 221 time-series data generation unit

123, 223 symmetry calculation unit

225 storage unit

227 step length calculation unit

330 display unit

What is claimed is:

1. A gait measurement system, comprising:
a data acquisition device configured to measure physical quantities related to pressures of left and right feet; and
a calculation device including
at least one memory storing instructions, and
at least one processor connected to the at least one memory and configured to execute the instructions to calculate a symmetry of walking using the physical quantities related to the pressures of the left and right feet.

2. The gait measurement system according to claim 1, wherein
the at least one processor of the calculation device is configured to execute the instructions to:
generate time-series data of pressure values using the physical quantities related to the pressures of the left and right feet; and
calculate the symmetry of the pressures of the left and right feet as the symmetry of walking using the time-series data of the pressure values of the left and right feet.

3. The gait measurement system according to claim 2, wherein
the at least one processor of the calculation device is configured to execute the instructions to
calculate the symmetry of walking using the relationship of maximum values of peaks relevant to each other between the left and right feet in one gait cycle among at least one peak appearing in each of the time-series data of pressure values of the left and right feet.

4. The gait measurement system according to claim 2, wherein
the at least one processor of the calculation device is configured to execute the instructions to
calculate the symmetry of walking using the relationship of maximum values of a plurality of peaks relevant to each other between the left and right feet in one gait cycle among at least one peak appearing in each of the time-series data of pressure values of the left and right feet.

5. The gait measurement system according to claim 3, wherein
the at least one processor of the calculation device is configured to execute the instructions to
calculate the symmetry of walking using at least one of a maximum value of a first peak at which a pressure applied from a heel of a pedestrian becomes maximum, a maximum value of a second peak at which a pressure applied from a toe of the pedestrian becomes maximum, and a maximum value of a dip appearing between the first peak and the second peak among at least one peak appearing each of the time-series data of the pressure values of the left and right feet.

6. The gait measurement system according to claim 5, wherein
the at least one processor of the calculation device is configured to execute the instructions to
calculate the symmetry of walking using the relationship of the maximum value of the first peak, the maximum value of the second peak, and the maximum value of the dip.

7. The gait measurement system according to claim 6, wherein
the at least one processor of the calculation device is configured to execute the instructions to:
store a regression model in which the symmetry of walking calculated using the relationship between the maximum value of the first peak, the maximum value of the second peak, and the maximum value of the dip is associated with the symmetry of the step length; and
calculate the symmetry of the step lengths from the symmetry of walking using the regression model and calculate the step lengths of the left and right feet using the calculated symmetry of the step lengths.

8. The gait measurement system according to claim 1 further comprising:
a display device configured to display information on the symmetry of walking.

9. A gait measurement method, comprising:
by a computer,
acquiring physical quantities related to pressures of left and right feet; and
calculating a symmetry of walking using the physical quantities related to the acquired pressures of the left and right feet.

10. A non-transitory program recording medium recorded with a program causing a computer to perform a process comprising:
acquiring physical quantities related to pressures of left and right feet; and
calculating a symmetry of walking using the physical quantities related to the pressures of the left and right feet.
